(19) United States
(12) Patent Application Publication
(10) Pub. No.: US 2005/0126576 A1
(43) Pub. Date: Jun. 16, 2005

(54) Protecting biological structures, including the great vessels, particularly during spinal surgery

(76) Inventor: Bret A. Ferree, Cincinnati, OH (US)

Correspondence Address: Gifford, Krass, Groh, Sprinkle,;Anderson & Citkowski, P.C., Suite 400, 280 N. Old Woodward Ave., Birmingham, MI, 48009-5394, US

(21) Appl. No.: 10/981,341

(22) Filed: Nov. 4, 2004

Related U.S. Application Data
(60) Provisional application No. 60/517,204, filed on Nov. 4, 2003.

(51) Int. Cl. A61F 5/37

(52) U.S. Cl. 128/846

(57) ABSTRACT

Natural and/or synthetic materials to form a strong barrier between the skeletal system and the great vessels. In the preferred embodiments, a natural or synthetic material is used to prevent scar tissue from forming around the vessels and/or to act as barrier placed between the vessels and the skeletal system, including the spine. Devices according to the invention may also be used over the dura and nerves following laminectomy procedures, between the sternum and the pericardium or heart following cardiac procedures, in intra-abdominal procedures such as intestinal or vascular surgery, over the brain in intra-cranial surgery, over the ovaries or other organs or tissues in the female genitourinary system, over the prostate or other organ or tissues in the male genitourinary system, or in other surgeries on humans or animals.

### REFERENCE TO RELATED APPLICATIONS

This application claims priority from U.S. Provisional Patent Application Ser. No. 60/517,204, filed Nov. 4, 2003, the entire content of which is incorporated herein by reference.

### FIELD OF THE INVENTION

This invention relates generally to surgical instruments and techniques and, in particular, to the use of natural and synthetic materials to protect one or more of the great vessels.

### BACKGROUND OF THE INVENTION

Revision anterior spinal surgery following a previous anterior spinal surgery is dangerous. The great vessels including the aorta, vena cava, iliac arteries, and iliac veins often become adherent to the operated area of the spine after the primary procedure. Frequently, the vessels must be mobilized from the anterior aspect of the spine during secondary anterior spinal procedures. Mobilizing vessels that are scared to the spine risks vessel injury. The thin walls of the veins make them particularly at risk of injury.

Prior art devices, such as that taught in U.S. Pat. No. 6,475,219 do not provide an adhesion free plane between the great vessels and the spine. The '219 device does not have a hollow, adhesion free space within the device. Adhesions would likely surround the '219 device. The '219 device would likely become adherent to the great vessels and the spine.

### SUMMARY OF THE INVENTION

This invention broadly uses natural and/or synthetic materials to form a strong barrier between the skeletal system and the great vessels. In the preferred embodiments, a natural or synthetic material is used to prevent scar tissue from forming around the vessels and/or to act as barrier placed between the vessels and the skeletal system, including the spine.

Devices according to the invention may also be used over the dura and nerves following laminectomy procedures, between the sternum and the pericardium or heart following cardiac procedures, in intra-abdominal procedures such as intestinal or vascular surgery, over the brain in intra-cranial surgery, over the ovaries or other organs or tissues in the female genitourinary system, over the prostate or other organ or tissues in the male genitourinary system, or in other surgeries on humans or animals.

### BRIEF DESCRIPTION OF THE DRAWINGS

FIG. 1A is an anterior view of the novel sleeve;

FIG. 1B is an anterior view of the novel sleeve wrapped around the vena cava and iliac veins;

FIG. 2A is an anterior view of an alternative embodiment of the invention;

FIG. 2B is an anterior view of the aorta, vena cava, iliac vessels, and the embodiment of the device drawn in FIG. 2A;

FIG. 2C is a cross section of the aorta, vena cava, and the embodiment of the device drawn in FIG. 2A;

FIG. 2D is a cross section of the aorta, vena cava, and the embodiment of the device drawn in FIG. 2C;

FIG. 2E is an anterior view of the great vessels, the embodiment of the device drawn in FIG. 2B, and the lumbar spine; and

FIG. 2F is an axial cross section of a lumbar vertebra, the great vessels, and the embodiment of the invention drawn in FIG. 2C.

### DETAILED DESCRIPTION OF THE INVENTION

According to one aspect of this invention, a sheet of material such as Dacron or Gortex is used protect the vessels during repeat surgeries. The high tensile strength of the material would enable surgeons to dissect between the material and the spine while protecting the vessels from injury. Natural tissues such as animal or human allograft aorta could alternatively be used. Metal or plastic sheets may also be placed between the vessels and the spine, and the barrier sheets may be attached to the spine or elsewhere to prevent migration.

Hydrogels, proteoglycans, hyaluronic acid, fibrinogen, or autograft fat may be placed between the vessels and the spine or between the spine and the implanted reinforcement membrane to decrease scar tissue formation. Other material that prevents scar tissue could also be used for this purpose. Furthermore, in situ curing polymers or sheets of silastic, as described in my U.S. Pat. No. 6,248,106, could be placed between the spine and the vessels.

Sheets of hydrogel are used in the preferred embodiments of the invention. The sheets may be attached to the vertebrae, ADR, or the vessels. The sheets could be reinforced with a nylon, gortex, or synthetic mesh such as Dacron. The hydrogel could be contained within a polymer balloon. Alternatively, the balloon could be filled with fluid.

Reinforcing devices according to the invention may also be wrapped around the vessels. For example, an allograft aorta-iliac sleeve could be wrapped around the vena cava and the iliac veins. The allograft sleeve could be reinforced with a synthetic mesh. A material the reduced scar tissue formation, such as Septra film, could be wrapped around the composite sleeve.

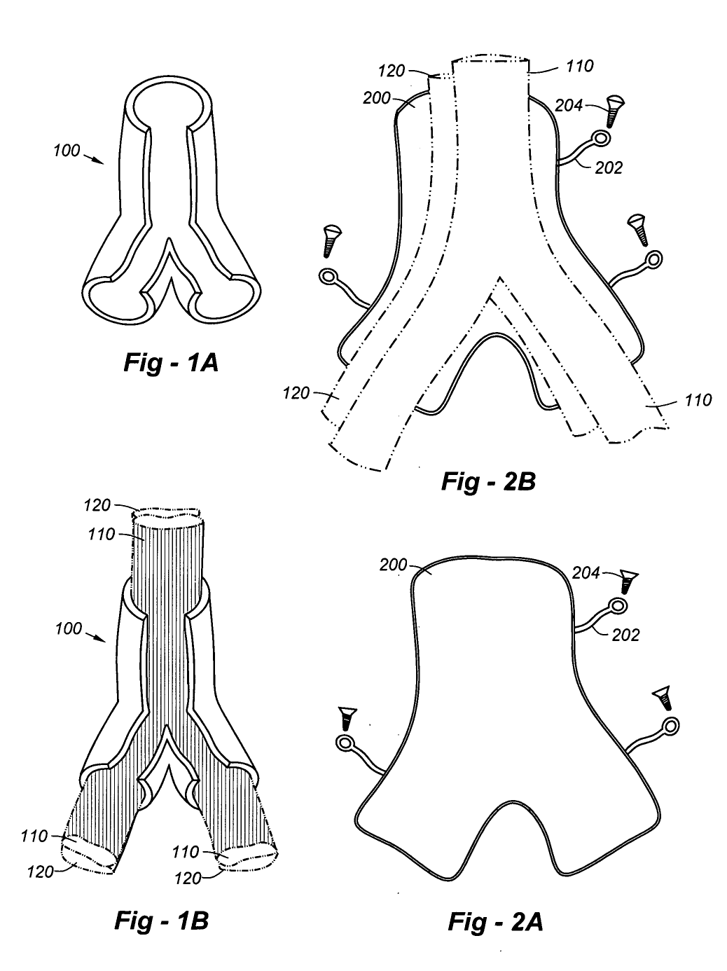

FIG. 1A is an anterior view of a sleeve constructed in accordance with this invention. FIG. 1B is an anterior view of the sleeve wrapped around the vena cava and iliac veins. The sleeve would preferably lie over the operated portion of the spine.

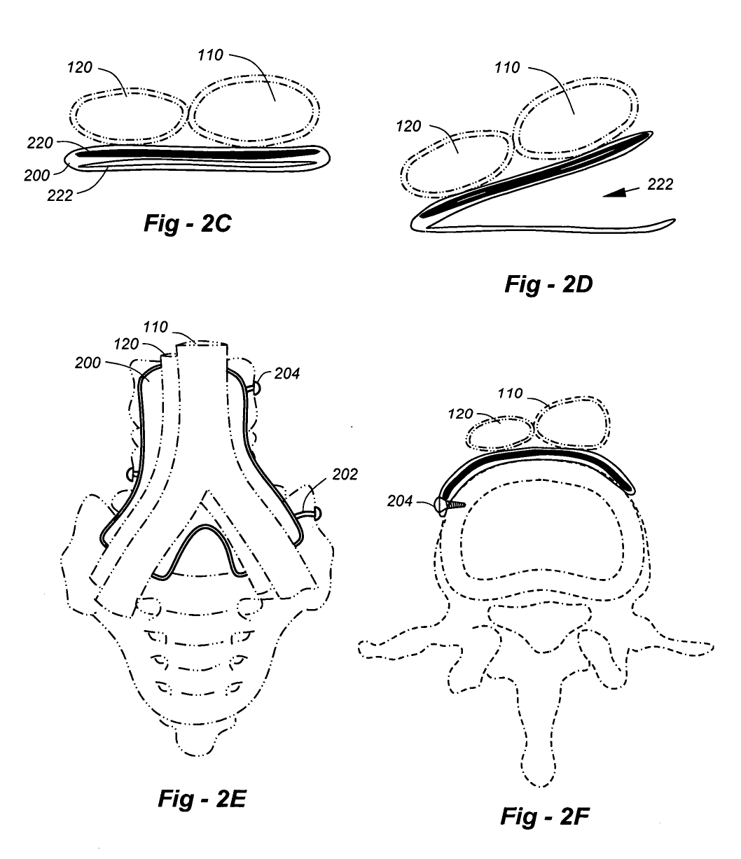

FIG. 2A is an anterior view of an alternative embodiment of the invention ion the form of a device 200 which is placed between the great vessels and the spine. The device may be tethered to the vertebrae, discs, artificial disc replacement(s), or soft tissues. Resorbable screws 204 may be placed through holes in tether components 202. Slack in the tether components would allow the device to side through a controlled range of motion.

The device 200 is preferably made of an elastomer, such as the elastomers used in cardiac balloons and a reinforcing mesh material, such as Dacron, Marlex, or nylon. The elastomer prevents scar tissue, or adhesions, from growing into the hollow interior of the device. Furthermore, the device is sealed to prevent adhesions from growing into the hollow interior of the device. A radiographic marker, such as a radio-opaque thread, may be incorporated into the periphery of the device. The device is preferably a color different than the color of the tissues that surround the spine. For example, the device could be made of purple or green material. The device may incorporate struts to prevent the device from wrinkling.

FIG. 2B is an anterior view of the aorta 110, vena cava 120, iliac vessels, and the embodiment of the device drawn in FIG. 2A. The device preferably lies between the great vessels and the anterior portion of the spine. FIG. 2C is a cross section of the aorta, vena cava, and the embodiment of the device drawn in FIG. 2A. FIG. 2D is a cross section of the aorta, vena cava, and the embodiment of the device drawn in FIG. 2C. The device has been cut lateral to the aorta. An adhesion free space 222 within the device allows easy dissection between the great vessels and the spine. The drawing illustrates retraction of the great vessels by separating the sides of the device through the opening within the device. The adhesion free space within my device allows surgeons to bypass adhesions between the device and the great vessels and between the device and the spine. The reinforced section 220 of the device 200 adjacent to the great vessels improves the tensile strength of the device, better facilitating retraction of the great vessels.

FIG. 2E is an anterior view of the great vessels, the embodiment of the device drawn in FIG. 2B, and the lumbar spine. Again, slack in the tether components may be used to allow the device to slide over the spine during spinal movement. FIG. 2F is an axial cross section of a lumbar vertebra, the great vessels, and the embodiment of the invention drawn in FIG. 2C.

The device may also be used over the dura and nerves following laminectomy procedures, between the sternum and the pericardium or heart following cardiac procedures, in intra-abdominal procedures such as intestinal or vascular surgery, over the brain in intra-cranial surgery, over the ovaries or other organs or tissues in the female genitourinary system, over the prostate or other organ or tissues in the male genitourinary system, or in other surgeries on humans or animals.

The device may be made of polyurethanes, such as polycarbonates and polyethers, such as Chronothane P 75A or P 55D (P-eth-PU aromatic, CT Biomaterials); Chronoflex C 55D, C 65D, C 80A, or C 93A (PC-PU aromatic, CT Biomaterials); Elast-Eon II 80A (Si-PU aromatic, Elastomedic); Bionate 55D/S or 80A-80A/S (PC-PU aromatic with S-SME, PTG); CarboSil-10 90A (PC-Si-PU aromatic, PTG); Tecothane TT-1055D or TT-1065D (P-eth-PU aromatic, Thermedics); Tecoflex EG-93A (P-eth-PU aliphatic, Thermedics); and Carbothane PC 3585A or PC 3555D (PC-PU aliphatic, Thermedics).

Preferably, the material is a segmented polyurethane, having a thickness ranging from about 5 to about 30 mils, more particularly about 10-11 mils. Examples of suitable materials include BIOSPAN-S (aromatic polyetherurethaneurea with surface modified end groups, Polymer Technology Group), CHRONOFLEX AR/LT (aromatic polycarbonate polyurethane with low-tack properties, CardioTech International), CHRONOTHANE B (aromatic polyether polyurethane, CardioTech International), CARBOTHANE PC (aliphatic polycarbonate polyurethane, Thermedics).

Devices according to this invention may reinforced with Goretex (W.T. Gore Company, Phoenix, Ariz.). Resorbable screws, staples, or other fastener devices may be obtained from Zimmer, Warsaw, Ind.

1. A device used to protect one or more great vessels, comprising:
a layer of barrier material, including:
an upper portion having a width corresponding to the diameter of a great vessel; and
a lower portion branching into two sections, each having a width corresponding to the diameter of a great vessel.

2. The device of claim 1, further including one or more tethers enabling the layer of barrier material to fastened to a biological structure to prevent migration.

3. The device of claim 1, wherein the barrier material includes a layer of reinforcement material.

4. A method of protecting the great vessels during spinal surgery, comprising the step of:
providing the device of claim 1; and
surgically placing the device between the spine and one or more of the great vessels.

5. The method of claim 4, further including the step of wrapping the device around one or more great vessels.
